(19) United States
(12) Patent Application Publication
(10) Pub. No.: US 2016/0025728 A1
(43) Pub. Date: Jan. 28, 2016

(54) BRAIN-SPECIFIC GENE SIGNATURE OF TUMOR CELLS

(71) Applicant: BOARD OF REGENTS, THE UNIVERSITY OF TEXAS SYSTEM, Austin, TX (US)

(72) Inventors: Isaiah J. FIDLER, Houston, TX (US); Sun Jin KIM, Houston, TX (US)

(73) Assignee: Board of Regents, The University of Texas System, Austin, TX (US)

(21) Appl. No.: 14/775,715

(22) PCT Filed: Mar. 13, 2014
(86) PCT No.: PCT/US2014/025799

§ 371 Date: Sep. 13, 2015

Related U.S. Application Data
(60) Provisional application No. 61/787,986, filed on Mar. 15, 2013.

(51) Int. Cl. G01N 33/574; A61B 6/03; A61B 6/00; A61B 5/055; C12Q 1/68; G06F 19/18

(52) U.S. Cl. CPC G01N 33/57407; C12Q 1/6886; G06F 19/18; A61B 6/501; A61B 5/055; A61B 6/032; A61B 6/037; C12Q 2600/158

(57) ABSTRACT

The present disclosure provides a method of detecting and diagnosing metastatic spread of cancer to the brain by analyzing the product of a gene signature. Gene signatures detailed herein are specific to the brain metastasis and not dependent upon the genetic signature of the primary tumor. Accordingly, disclosed method can be used to guide therapeutic intervention and further diagnostic analysis.

This application claims the benefit of U.S. Provisional Patent Application No. 61/787,986, filed Mar. 15, 2013, the entirety of which is incorporated herein by reference.

### BACKGROUND OF THE INVENTION

1. Field of the Invention

The present invention relates generally to the field of cancer biology. More particularly, it concerns methods for the early detection of brain metastases.

2. Description of Related Art

Currently there are no early stage diagnostic tests to determine primary neoplasm and/or metastatic spread of cancer. Under current therapy, tumors and/or metastasis often times cannot be diagnosed until the lesion is large enough to be imaged. Thus, there remains a need for sensitive diagnostics to detect cancer and, in particular, cancer metastasis.

### SUMMARY OF THE INVENTION

The present disclosure provides a method of detecting and diagnosing brain tumors and/or metastatic spread of cancer to the brain by analyzing the product of a gene (or protein) signature, wherein said signature is specific to the brain metastasis and not dependent upon the signature of the primary tumor. In certain aspects, a method comprises a blood test to identify proteins that are expressed from the brain-specific gene signature to identify early stage primary tumors and/or metastatic disease in the brain.

In a first embodiment, the present disclosure provides a method of detecting if a subject is at risk for a brain cancer or cancer brain metastasis, comprising (a) obtaining a biological sample from the subject (e.g., a blood or serum sample); (b) measuring the expression level of one or more genes in the sample selected from the group consisting of GRIA2, C12orf48, GRM3, CRYAB, CELSR3 (cadherin-P), SREBF1, LOC100008589, CTNNB1, STAT3, CD81, FTHL7, IL8, ACTB and one of the genes selected from Table 1; and (c) identifying the subject as at risk or not at risk for a brain cancer or cancer brain metastasis based on the expression level of said genes. In some specific aspects, a method comprises measuring the expression level of one or more of GRIA2, C12orf48, GRM3, CRYAB, CELSR3 (cadherin-P), SREBF1, LOC100008589, CTNNB1, STAT3, CD81, FTHL7, IL8 or ACTB. Thus, in some aspects, the subject is identified as at risk for a brain cancer or cancer brain metastasis if the expression of one or more of the genes is elevated in the sample as compared to a reference.

In some aspects, the subject has or is diagnosed with a cancer. For example, the cancer can be a breast cancer, lung cancer, head & neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In one aspect, the subject has been previously treated for a cancer. In some aspects, the subject had a cancer that has been previously treated, such as a subject who has had a tumor surgically removed.

In one aspect, the method further comprises measuring the expression level of at least 2 of said genes, wherein elevated expression of the genes compared to a reference indicates that the subject as at risk for a brain cancer or cancer brain metastasis. In another aspect, the method further comprises measuring the expression level of at least 3, 4, 5, 6, 7, 8, 9, 10, 11 or 12 of said genes. In yet a further aspect, the method further comprises measuring the expression level of two, three or more of the CRYAB, CELSR3, SREBF1, CD81, FTHL7, ACTB, C12orf48, LOC100008589, or IL8 genes. In still another aspect, the method further comprises measuring the expression level of two, three or more of the CRYAB, CELSR3, SREBF1, CD81, FTHL7, or ACTB genes.

In some aspects, identifying the subject as at risk or not at risk for a brain cancer or cancer brain metastasis further comprises correlating the measured expression level(s) with a risk for cancer brain metastasis. In another aspect, identifying the subject as at risk or not at risk for a cancer brain metastasis further comprises analysis of the measured expression level(s) using an algorithm. In some cases, a correlation or analysis may be performed by a computer.

In a further aspect, the method further comprises (b) measuring the expression level of the gene(s) in the sample and measuring the expression level of the genes in a reference sample; and (c) identifying the subject as at risk or not at risk for a brain cancer or cancer brain metastasis by comparing the expression level of the gene(s) in the sample from the subject to the expression level of the genes in the reference sample.

In some aspects, measuring expression of said gene(s) comprises measuring protein expression levels (e.g., protein expression levels inn blood or serum). Protein expression levels may be measured, for example, by performing a Western blot, an ELISA or binding to an antibody array. In another aspect, measuring expression of said genes comprises measuring RNA expression levels. RNA expression levels may be measured by performing RT-PCR, Northern blot or an array hybridization.

In further aspects, a method further comprises reporting whether the subject is at risk or not at risk for a brain cancer or cancer brain metastasis. In one aspect, reporting may comprise preparing a written, oral or electronic report. In some cases, reporting may further comprise providing the report to the patient, a doctor, a hospital or an insurance company.

In a further embodiment, the present disclosure provides a method of treating a subject comprising selecting a subject identified as at risk for a brain cancer or cancer brain metastasis in accordance with the embodiments; and administering an anti-cancer therapy the subject. For example, a method can comprise (a) obtaining the expression level of one or more genes selected from the group consisting of GRIA2, C12orf48, GRM3, CRYAB, CELSR3 (cadherin-P), SREBF1, LOC100008589, CTNNB1, STAT3, CD81, FTHL7, IL8, and ACTB in a sample from the subject; (b) selecting a subject having a risk for a cancer brain metastasis based on the expression level of said gene(s); and (c) treating the selected subject with an anti-cancer therapy. In certain aspects, the anti-cancer therapy is a chemotherapy, a radiation therapy, a hormonal therapy, a targeted therapy, an immunotherapy or a surgical therapy. In one aspect, the anti-cancer therapy is targeted to the brain. In some specific aspects, the anticancer therapy can be a therapy targeted to the cancer, such as a therapy targeted to a GRIA2, C12orf48, GRM3, CRYAB, CELSR3 (cadherin-P), SREBF1, LOC100008589, CTNNB1, STAT3, CD81, FTHL7, IL8, or ACTB protein expressing cell.

In still a further embodiment, the present disclosure provides a method of selecting a subject for a diagnostic procedure comprising selecting a subject identified as at risk for a brain cancer or cancer brain metastasis in accordance with the embodiments; and performing a diagnostic procedure on the selected on the subject. For example, a method can comprise (a) obtaining the expression level of one or more genes selected from the group consisting of GRIA2, C12orf48, GRM3, CRYAB, CELSR3 (cadherin-P), SREBF1, LOC100008589, CTNNB1, STAT3, CD81, FTHL7, IL8, and ACTB in a sample from the subject; (b) selecting a subject having a risk for a brain cancer or cancer brain metastasis based on the expression level of said gene(s); and (c) performing a diagnostic procedure on the subject on the subject. In one aspect, the diagnostic procedure comprises imaging of the head. In one aspect, the imaging is a X-ray, CT, MRI or PET imaging.

In one embodiment, the present disclosure provides a tangible computer-readable medium comprising computer-readable code that, when executed by a computer, causes the computer to perform operations comprising (a) receiving information corresponding to a level of expression of GRIA2, C12orf48, GRM3, CRYAB, CELSR3 (cadherin-P), SREBF1, LOC100008589, CTNNB1, STAT3, CD81, FTHL7, IL8, or ACTB gene in a sample from a subject; and (b) determining a relative level of expression of one ore more of said genes compared to a reference level, wherein altered expression of one ore more of said genes compared to a reference level indicates that the subject is at risk of having a cancer brain metastasis.

In one aspect, the tangible computer-readable medium further comprises receiving information corresponding to a reference level of expression of GRIA2, C12orf48, GRM3, CRYAB, CELSR3 (cadherin-P), SREBF1, LOC100008589, CTNNB1, STAT3, CD81, FTHL7, IL8, or ACTB in a sample from a subject without brain metastasis. In one aspect, the reference level is stored in said tangible computer-readable medium.

In one aspect, the tangible computer-readable medium further comprises computer-readable code that, when executed by a computer, causes the computer to perform one or more additional operations comprising: sending information corresponding to the relative level of expression of one or more of said genes to a tangible data storage device.

In one aspect, receiving information comprises receiving from a tangible data storage device information corresponding to a level of expression of one or more of said gene in a sample from a subject. In another aspect, the receiving information further comprises receiving information corresponding to a level of expression of at least 2, 3, 4, 5, 6, 7, 8, 9, 10, 11 or 12 of said genes in a sample from a subject. In one aspect, the computer-readable code, when executed by a computer, causes the computer to perform operations further comprising (c) calculating a diagnostic score for the sample, wherein the diagnostic score is indicative of the probability that the sample is from a subject having a cancer brain metastasis.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Antibody microarray study 1. Left: imaging of mice implanted with MDA231 cells by mammary fat pad or internal carotid injection. Right: serum and antibody microarray processing schematic.

FIG. 2. Antibody microarray study 2. Imaging of mice implanted with MDA231 or PC14 cells by internal carotid injection.

FIG. 3. Antibody microarray study 3. Imaging of mice implanted with LN220 cells by stereotactic injection.

### DESCRIPTION OF ILLUSTRATIVE EMBODIMENTS

When cancer cells grow in or metastasize to the brain, in addition to their tumor specific gene cluster, the cancer cells up-regulate a new set of gene clusters regulated by their organ environment. Accordingly, brain cancer and brain metastatic disease contains at least two specific gene signature: (1) the signature identifying the primary cancer type and (2) a signature that identifies the cancer as having metastasized to the brain. The brain-specific gene signature does not vary based on the primary tumor type or primary tumor gene signature and is universal for all metastases to the brain.

As such, diagnostic tests are contemplated that detect brain specific metastases as well as primary brain cancers. Such diagnostic tests are preferably blood tests to identify the proteins that are expressed from the brain-specific gene signature and can be used to identify early stage metastatic disease. Also contemplated are therapies (e.g., drugs, vaccines, antibodies, etc.) that target the brain-specific gene signature of metastatic cells that can be used as a universal treatment of metastatic disease in the brain regardless of the origin of the metastasis.

### I. BIOMARKER DETECTION

The expression of biomarkers or genes may be measured by a variety of techniques that are well known in the art. Quantifying the levels of the messenger RNA (mRNA) of a biomarker may be used to measure the expression of the biomarker. Alternatively, quantifying the levels of the protein product of a biomarker may be used to measure the expression of the biomarker. Additional information regarding the methods discussed below may be found in Ausubel et al. (2003) or Sambrook et al. (1989). One skilled in the art will know which parameters may be manipulated to optimize detection of the mRNA or protein of interest.

In some embodiments, said obtaining expression information may comprise RNA quantification, e.g., cDNA microarray, quantitative RT-PCR, in situ hybridization, Northern blotting or nuclease protection. Said obtaining expression information may comprise protein quantification, e.g., protein quantification comprises immunohistochemistry, an ELISA, a radioimmunoassay (RIA), an immunoradiometric assay, a fluoroimmunoassay, a chemiluminescent assay, a bioluminescent assay, a gel electrophoresis, a Western blot analysis, a mass spectrometry analysis, or a protein microarray.

A nucleic acid microarray may be used to quantify the differential expression of a plurality of biomarkers. Microarray analysis may be performed using commercially available equipment, following manufacturer's protocols, such as by using the Affymetrix GeneChip® technology (Santa Clara, Calif.) or the Microarray System from Incyte (Fremont, Calif.). For example, single-stranded nucleic acids (e.g., cDNAs or oligonucleotides) may be plated, or arrayed, on a microchip substrate. The arrayed sequences are then hybridized with specific nucleic acid probes from the cells of interest. Fluorescently labeled cDNA probes may be generated through incorporation of fluorescently labeled deoxynucleotides by reverse transcription of RNA extracted from the cells of interest. Alternatively, the RNA may be amplified by in vitro transcription and labeled with a marker, such as biotin. The labeled probes are then hybridized to the immobilized nucleic acids on the microchip under highly stringent conditions. After stringent washing to remove the non-specifically bound probes, the chip is scanned by confocal laser microscopy or by another detection method, such as a CCD camera. The raw fluorescence intensity data in the hybridization files are generally preprocessed with the robust multichip average (RMA) algorithm to generate expression values.

Quantitative real-time PCR (qRT-PCR) may also be used to measure the differential expression of a plurality of biomarkers. In qRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified.

A non-limiting example of a fluorescent reporter probe is a TaqMan® probe (Applied Biosystems, Foster City, Calif.). The fluorescent reporter probe fluoresces when the quencher is removed during the PCR extension cycle. Multiplex qRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, qRT-PCR may be performed using a reference standard. The ideal reference standard is expressed at a constant level among different tissues, and is unaffected by the experimental treatment. Suitable reference standards include, but are not limited to, mRNAs for the housekeeping genes glyceraldehyde-3-phosphate-dehydrogenase (GAPDH) and β-actin. The level of mRNA in the original sample or the fold change in expression of each biomarker may be determined using calculations well known in the art.

Immunohistochemical staining may also be used to measure the differential expression of a plurality of biomarkers. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined This measurement, along with a quantification of the intensity of staining, may be used to generate an expression value for the biomarker.

An enzyme-linked immunosorbent assay, or ELISA, may be used to measure the differential expression of a plurality of biomarkers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of a plurality of biomarkers. For this, a plurality of antibodies is arrayed and covalently attached to the surface of the microarray or biochip. A protein extract containing the biomarker proteins of interest is generally labeled with a fluorescent dye or biotin. The labeled biomarker proteins are incubated with the antibody microarray. After washes to remove the unbound proteins, the microarray is scanned. The raw fluorescent intensity data may be converted into expression values using means known in the art.

Luminex multiplexing microspheres may also be used to measure the differential expression of a plurality of biomarkers. These microscopic polystyrene beads are internally color-coded with fluorescent dyes, such that each bead has a unique spectral signature (of which there are up to 100). Beads with the same signature are tagged with a specific oligonucleotide or specific antibody that will bind the target of interest (i.e., biomarker mRNA or protein, respectively). The target, in turn, is also tagged with a fluorescent reporter. Hence, there are two sources of color, one from the bead and the other from the reporter molecule on the target. The beads are then incubated with the sample containing the targets, of which up to 100 may be detected in one well. The small size/surface area of the beads and the three dimensional exposure of the beads to the targets allows for nearly solution-phase kinetics during the binding reaction. The captured targets are detected by high-tech fluidics based upon flow cytometry in which lasers excite the internal dyes that identify each bead and also any reporter dye captured during the assay. The data from the acquisition files may be converted into expression values using means known in the art.

In situ hybridization may also be used to measure the differential expression of a plurality of biomarkers. This method permits the localization of mRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an mRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple mRNAs may be detected simultaneously, provided each antisense probe has a distinguishable label. The hybridized tissue array is generally scanned under a microscope. Because a sample of tissue from a subject with cancer may be heterogeneous, i.e., some cells may be normal and other cells may be cancerous, the percentage of positively stained cells in the tissue may be determined This measurement, along with a quantification of the intensity of staining, may be used to generate an expression value for each biomarker.

In a further embodiment, the marker level may be compared to the level of the marker from a control, wherein the control may comprise one or more tumor samples taken from one or more patients determined as having a certain metastatic tumor or not having a certain metastatic tumor, or both.

The control may comprise data obtained at the same time (e.g., in the same hybridization experiment) as the patient's individual data, or may be a stored value or set of values, e.g., stored on a computer, or on computer-readable media. If the latter is used, new patient data for the selected marker(s), obtained from initial or follow-up samples, can be compared to the stored data for the same marker(s) without the need for additional control experiments.

### II. DEFINITIONS

As used herein, “obtaining a biological sample” or “obtaining a blood sample” refer to receiving a biological or blood sample, e.g., either directly or indirectly. For example, in some embodiments, the biological sample, such as a blood sample or a sample containing peripheral blood mononuclear cells (PBMC), is directly obtained from a subject at or near the laboratory or location where the biological sample will be analyzed. In other embodiments, the biological sample may be drawn or taken by a third party and then transferred, e.g., to a separate entity or location for analysis. In other embodiments, the sample may be obtained and tested in the same location using a point-of care test. In these embodiments, said obtaining refers to receiving the sample, e.g., from the patient, from a laboratory, from a doctor's office, from the mail, courier, or post office, etc. In some further aspects, the method may further comprise reporting the determination to the subject, a health care payer, an attending clinician, a pharmacist, a pharmacy benefits manager, or any person that the determination may be of interest.

By “subject” or “patient” is meant any single subject for which therapy or diagnostic test is desired. This case the subjects or patients generally refer to humans. Also intended to be included as a subject are any subjects involved in clinical research trials not showing any clinical sign of disease, or subjects involved in epidemiological studies, or subjects used as controls.

As used herein, “increased expression” refers to an elevated or increased level of expression in a cancer sample relative to a suitable control (e.g., a non-cancerous tissue or cell sample, a reference standard), wherein the elevation or increase in the level of gene expression is statistically significant (p<0.05). Whether an increase in the expression of a gene in a cancer sample relative to a control is statistically significant can be determined using an appropriate t-test (e.g., one-sample t-test, two-sample t-test, Welch's t-test) or other statistical test known to those of skill in the art. Genes that are overexpressed in a cancer can be, for example, genes that are known, or have been previously determined, to be overexpressed in a cancer.

As used herein, “decreased expression” refers to a reduced or decreased level of expression in a cancer sample relative to a suitable control (e.g., a non-cancerous tissue or cell sample, a reference standard), wherein the reduction or decrease in the level of gene expression is statistically significant (p<0.05). In some embodiments, the reduced or decreased level of gene expression can be a complete absence of gene expression, or an expression level of zero. Whether a decrease in the expression of a gene in a cancer sample relative to a control is statistically significant can be determined using an appropriate t-test (e.g., one-sample t-test, two-sample t-test, Welch's t-test) or other statistical test known to those of skill in the art. Genes that are underexpressed in a cancer can be, for example, genes that are known, or have been previously determined, to be underexpressed in a cancer.

The term “antigen binding fragment” herein is used in the broadest sense and specifically covers intact monoclonal antibodies, polyclonal antibodies, multispecific antibodies (e.g., bispecific antibodies) formed from at least two intact antibodies, and antibody fragments.

The term “primer,” as used herein, is meant to encompass any nucleic acid that is capable of priming the synthesis of a nascent nucleic acid in a template-dependent process. Primers may be oligonucleotides from ten to twenty and/or thirty base pairs in length, but longer sequences can be employed. Primers may be provided in double-stranded and/or single-stranded form, although the single-stranded form is preferred.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Brain-Specific Metastasis Signature

Different tumor cell lines producing metastases in the brain were analyzed for a common gene signature. The tumor cell lines used were PC14 (lung adenocarcinoma), A375 (melanoma), PC3 (prostate), MDA-MB-231 (breast), KM12 (colon), and SN12 (kidney). Two hundred ten genes were found to correlate with a brain-specific gene signature (Table 1).

TABLE 1
Brain metastasis gene signature - differentially expressed genes.
Brain

| Serial # | DEFINITION |
|---|---|
| 1. | zo30f03. s1 Stratagene colon (#937204) Homo sapiens eDNA clone IMAGE: |
| | 588413 3, mRNA sequence |
| 2. | Homo sapiens CaM kinase-like vesicle-associated (CAMKV), mRNA. |
| 3. | Homo sapiens bassoon (presynaptic cytomatrix protein) (BSN), mRNA. |
| 4. | Homo sapiens glutamate receptor, ionotropic, AMPA 2 (GR IA2), mRNA. |
| 5. | Homo sapiens calmodulin binding transcription activator 1 (CAMTA1), |
| | mRNA. |
| 6. | Homo sapiens SPARC-Iike 1 (mast9, hevin) (SPARCL1), mRNA. |
| 7. | Homo sapiens CAP-GLY domain containing linker protein 3 (CLIP3), mRNA. |
| 8. | Homo sapiens reticulon 1 (RTN1), transcript variant 3, mRNA. |
| 9. | Homo sapiens phosphatase and actin regulator 1 (PHACTR1), mRNA. |
| 10. | Homo sapiens synaptotagmin XI (SYT11), mRNA. |
| 11. | Homo sapiens Kv channel interacting protein 4 (KCNIP4), transcript variant 6, |
| | mRNA. |
| 12. | Homo sapiens myelin basic protein (MBP), transcript variant 7, mRNA. |
| 13. | Homo sapiens myelin basic protein (MBP), transcript variant 3, mRNA. |
| 14. | Homo sapiens junctophilin 4 (.JPH4), mRNA. |
| 15. | Homo sapiens BR serine/threonine kinase 1 (BRSK1), mRNA. |
| 16. | Homo sapiens eDNA FL.J37610 fis, clone BRCOC2011398 |
| 17. | Homo sapiens solute carrier family 32 (GABA vesicular transporter), member |
| | 1 (SLC32A 1), mRNA. |
| 18. | Homo sapiens fasciculation and elongation protein zeta 1 (zygin I) (FEZ1), |
| | transcript variant 1, mRNA. |
| 19. | Homo sapiens protein tyrosine phosphatase, receptor type, D (PTPRD), |
| | transcript variant 4, mRNA. |
| 20. | Homo sapiens synaptic vesicle glycoprotein 2A (SV2A), mRNA. |
| 21. | Homo sapiens ring finger protein 165 (RNF165), mRNA. |
| 22. | Homo sapiens ephrin-83 (EFNB3), mRNA. |
| 23. | Homo sapiens synaptosomal-associated protein, 25 kDa (SNAP25), transcript |
| | variant 2, mRNA. |
| 24. | Homo sapiens RAS-Iike, family 11, member B (RASL11B), mRNA. |
| 25. | Homo sapiens glycoprotein M6B (GPM6B), transcript variant 1, mRNA. |
| 26. | Homo sapiens cornichon homolog 2 (Drosophila) (CNIH2), mRNA. |
| 27. | Homo sapiens platelet-derived growth factor alpha polypeptide (PDGFA), |
| | transcript variant 2, mRNA. |
| 28. | Homo sapiens chromosome 12 open reading frame 48 (C12orf48), mRNA. |
| 29. | Homo sapiens potassium voltage-gated channel, shaker-related subfamily, |
| | member 1 (episodic ataxia with myokymia) (KCNA1), mRNA. |
| 30. | Homo sapiens leucine rich repeat and lg domain containing 1 (LING01), |
| | mRNA. |
| 31. | Homo sapiens proteolipid protein 1 (Pelizaeus-Merzbacher disease, spastic |
| | paraplegia 2, uncomplicated) (PLP1), |
| | transcript variant 1, mRNA. |
| 32. | Homo sapiens ataxin 2-binding protein 1 (A2BP1), transcript variant 1, |
| | mRNA. |
| 33. | Homo sapiens ataxin 2-binding protein 1 (A2BP1), transcript variant 1, |
| | mRNA. |
| 34. | Homo sapiens ELAV (embryonic lethal, abnormal vision, Drosophila)-like 4 |
| | (Hu antigen D) (ELAVL4), mRNA. |
| 35. | Homo sapiens glycoprotein M6B (GPM6B), transcript variant 1, mRNA. |
| 36. | Homo sapiens calcium/calmodulin-dependent protein kinase (CaM kinase) II |
| | beta (CAMK2B), transcript variant 4, mRNA. |
| 37. | PREDICTED: Homo sapiens similar to bruno-like 4, RNA binding protein |
| | (LOC644278), mRNA. |
| 38. | Homo sapiens glutamate receptor, metabotropic 3 (GRM3), mRNA. |
| 39. | Homo sapiens transmembrane protein 178 (TMEM178), mRNA. |
| 40. | Homo sapiens synaptosomal-associated protein, 25 kDa (SNAP25), transcript |
| | variant 2, mRNA. |
| 41. | PREDICTED: Homo sapiens similar to protein phosphatase 1 regulatory |
| | subunit 148 (LOC648343), mRNA. |
| 42. | tt27g09. x1 NCI_CGAP_GC6 Homo sapiens eDNA clone IMAGE: 2242048 3, |
| | mRNA sequence |
| 43. | Homo sapiens protein tyrosine phosphatase, receptor-type, Z polypeptide 1 |
| | (PTPRZ1), mRNA. |
| 44. | Homo sapiens crystallin, alpha B (CRYAB), mRNA. |
| 45. | Homo sapiens eukaryotic translation elongation factor 1 alpha 2 (EEF1A2), |
| | mRNA. |
| 46. | Homo sapiens chromosome 14 open reading frame 4 (C14orf4), mRNA. |
| 47. | Homo sapiens guanine nucleotide binding protein (G protein), alpha activating |
| | activity polypeptide 0 (GNA01), 47 transcript variant 2, mRNA. |
| 48. | Homo sapiens EPH receptor A5 (EPHA5), transcript variant 1, mRNA. |
| 49. | Homo sapiens cytoplasmic polyadenylation element binding protein 3 |
| | (CPEB3), mRNA. |
| 50. | Homo sapiens neurotensin receptor 2 (NTSR2), mRNA. |
| 51. | Homo sapiens OTU domain, ubiquitin aldehyde binding 1 (OTUB1), transcript |
| | variant 2, transcribed RNA. |
| 52. | Homo sapiens estrogen-related receptor gamma (ESRRG), transcript variant 2, |
| | mRNA. |
| 53. | Homo sapiens teashirt zinc finger homeobox 1 (TSHZ1), mRNA. |
| 54. | Homo sapiens early growth response 1 (EGR1), mRNA. |
| 55. | Homo sapiens sal-like 2 (Drosophila) (SALL2), mRNA. |
| 56. | Homo sapiens presenilin enhancer 2 homolog (C. elegans) (PSENEN), mRNA. |
| 57. | Homo sapiens cadherin, EGF LAG seven-pass G-type receptor 3 (flamingo |
| | homolog, Drosophila) (CELSR3), mRNA. |
| 58. | Homo sapiens eDNA: FLJ21333 fis, clone COL02535 |
| 59. | Homo sapiens acyi-CoA synthetase long-chain family member 6 (ACSL6), |
| | transcript variant 2, mRNA. |
| 60. | PREDICTED: Homo sapiens similar to Translationally-controlled tumor |
| | protein (TCTP) (p23) (Histamine-releasing factor) (HRF) (Fortilin) |
| | (LOC643870), mRNA. |
| 61. | Homo sapiens SW I/SNF related, matrix associated, actin dependent regulator |
| | of chromatin, subfamily a, member 2 (SMARCA2), transcript variant 1, |
| | mRNA. |
| 62. | Homo sapiens dachshund homolog 1 (Drosophila) (DACH1), transcript variant |
| | 2, mRNA. |
| 63. | Homo sapiens leucine rich repeat and lg domain containing 2 (LING02), |
| | mRNA. |
| 64. | Homo sapiens myotubularin related protein 3 (MTMR3), transcript variant 3, |
| | mRNA. |
| 65. | Homo sapiens vacuolar protein sorting 53 homolog (S. cerevisiae) (VPS53), |
| | mRNA. |
| 66. | Homo sapiens glucocorticoid induced transcript 1 (GLCCI1), mRNA. |
| 67. | Homo sapiens vacuolar protein sorting 37 homolog D (S. cerevisiae) |
| | (VPS37D), mRNA. |
| 68. | Homo sapiens trinucleotide repeat containing 4 (TNRC4), mRNA. |
| 69. | Homo sapiens zinc finger and BTB domain containing 20 (ZBTB20), mRNA. |
| 70. | Homo sapiens TBC1 domain family, member 19 (TBC1D19), mRNA. |
| 71. | Homo sapiens potassium voltage-gated channel, Shab-related subfamily, |
| | member 2 (KCNB2), mRNA. |
| 72. | Homo sapiens single stranded DNA binding protein 3 (SSBP3), transcript |
| | variant 2, mRNA. |
| 73. | Homo sapiens brevican (BCAN), transcript variant 1, mRNA. |
| 74. | Homo sapiens centaurin, gamma 3 (CENTG3), transcript variant 2, mRNA. |
| 75. | Homo sapiens neuroplastin (NPTN), transcript variant beta, mRNA. |
| 76. | Homo sapiens ephrin-82 (EFNB2), mRNA. |
| 77. | PREDICTED: Homo sapiens similar to 60S ribosomal protein L27a |
| | (LOC391124), mRNA. |
| 78. | PREDICTED: Homo sapiens similar to NADH dehydrogenase (ubiquinone) 1 |
| | alpha subcomplex, 3, 9 kDa (LOC644482), mRNA. |
| 79. | Homo sapiens mRNA; cDNA DKFZp686F09166 (from clone |
| | DKFZp686F09166) |
| 80. | Homo sapiens family with sequence similarity 5, member C (FAM5C), |
| | mRNA. |
| 81. | Homo sapiens cytoplasmic polyadenylation element binding protein 2 |
| | (CPEB2), transcript variant A, mRNA. |
| 82. | PREDICTED: Homo sapiens hypothetical LOC441763 (LOC441763), mRNA. |
| 83. | Homo sapiens sterol regulatory element binding transcription factor 1 |
| | (SREBF1), transcript variant 2, mRNA. |
| 84. | Homo sapiens neurturin (NRTN), mRNA. |
| 85. | Homo sapiens syntaxin 16 (STX16), transcript variant 1, mRNA. |
| 86. | Homo sapiens phosphoglycerate kinase 2 (PGK2), mRNA. |
| 87. | Homo sapiens nuclear receptor binding protein 1 (NRBP1), mRNA. |
| 88. | Homo sapiens gametogenetin binding protein 2 (GGNBP2), mRNA. |
| 89. | Homo sapiens spermatid perinuclear RNA binding protein (STRBP), mRNA. |
| 90. | Homo sapiens stathmin-like 2 (STMN2), mRNA. |
| 91. | Homo sapiens F-box and leucine-rich repeat protein 5 (FBX L5), transcript |
| | variant 1, mRNA. |
| 92. | Homo sapiens AU RNA binding protein/enoyl-Coenzyme A hydratase (AUH), |
| | nuclear gene encoding mitochondrial protein, mRNA. |
| 93. | Homo sapiens chromosome 6 open reading frame 166 (C6orf166), mRNA. |
| 94. | Homo sapiens chromosome 11 open reading frame 56 (C11orf56), transcript |
| | variant 2, mRNA. |
| 95. | Homo sapiens serine threonine kinase 39 (STE20/SPS1 homolog, yeast) |
| | (STK39), mRNA. |
| 96. | PREDICTED: Homo sapiens similar to H3 histone, family 38 (LOC347376), |
| | mRNA. |
| 97. | Homo sapiens isocitrate dehydrogenase 3 (NAD+) gamma (IDH3G), nuclear |
| | gene encoding mitochondrial protein, transcript variant 2, mRNA. |
| 98. | Homo sapiens chromosome 19 open reading frame 6 (C19orf6), transcript |
| | variant 1, mRNA. |
| 99. | Homo sapiens crystallin, zeta (quinone reductase)-like 1 (CRYZL1), mRNA. |
| 100. | Homo sapiens TSC22 domain family, member 1 (TSC22D1), transcript variant |
| | 2, mRNA. |
| 101. | Homo sapiens v-ski sarcoma viral oncogene homolog (avian) (SKI), mRNA. |
| 102. | Homo sapiens CUG triplet repeat, RNA binding protein 1 (CUGBP1), |
| | transcript variant 3, mRNA. |
| 103. | Homo sapiens RNA-binding region (RNP1, RRM) containing 2 (RNPC2), |
| | transcript variant 3, mRNA. |
| 104. | Homo sapiens Ewing sarcoma breakpoint region 1 (EWSR1), transcript variant |
| | EWS, mRNA. |
| 105. | Homo sapiens glutamate receptor, ionotropic N-methyl D-aspartate-associated |
| | protein 1 (glutamate binding) (GRINA), transcript variant 1, mRNA. |
| 106. | Homo sapiens chromosome 6 open reading frame 125 (C6orf125), mRNA. |
| 107. | PREDICTED: Homo sapiens hypothetical LOC388588, transcript variant 2 |
| | (LOC388588), mRNA. |
| 108. | Homo sapiens protein phosphatase 2 (formerly 2A), regulatory subunit B. |
| | alpha isoform (PPP2R2A), mRNA. |
| 109. | PREDICTED: Homo sapiens hypothetical LOC145853 (LOC145853), mRNA. |
| 110. | Homo sapiens ring finger protein 44 (RNF44), mRNA. |
| 111. | Homo sapiens TLC domain containing 1 (TLCD1), mRNA. |
| 112. | Homo sapiens methylenetetrahydrofolate dehydrogenase (NADP+ dependent) |
| | 1, methenyltetrahydrofolate cyclohydrolase, formyltetrahydrofolate synthetase |
| | (MTHFD1), mRNA. |
| 113. | Homo sapiens nuclear receptor interacting protein 1 (NRIP1), mRNA. |
| 114. | Homo sapiens casein kinase 2, alpha prime polypeptide (CSNK2A2), mRNA. |
| 115. | Homo sapiens HLA-B associated transcript 2 (BAT2), mRNA. |
| 116. | Homo sapiens immediate early response 5-like (IER5L), mRNA. |
| 117. | Homo sapiens 28S ribosomal RNA (LOC100008589). |
| 118. | Homo sapiens general transcription factor IIH, polypeptide 4, 52 kDa |
| | (GTF2H4), mRNA. |
| 119. | Homo sapiens frizzled homolog 7 (Drosophila) (FZD7), mRNA. |
| 120. | PREDICTED: Homo sapiens SH2 domain containing 5 (SH2D5), mRNA. |
| 121. | Homo sapiens TAF10 RNA polymerase II, TATA box binding protein (TBP)- |
| | associated factor, 30 kDa (TAF10), mRNA. |
| 122. | Homo sapiens TSC22 domain family, member 1 (TSC22D1), transcript variant |
| | 2, mRNA. |
| 123. | Homo sapiens ankyrin repeat and KH domain containing 1 (ANKHD1), |
| | transcript variant 1, mRNA. |
| 124. | Homo sapiens cDNA: FLJ22720 fis, clone HSI14320 |
| 125. | Homo sapiens transmembrane protein 150 (TMEM150), transcript variant 1, |
| | mRNA. |
| 126. | Homo sapiens catenin (cadherin-associated protein), beta 1, 88 kDa (CTNNB1), |
| | mRNA. |
| 127. | Homo sapiens glycoprotein, synaptic 2 (GPSN2), mRNA. |
| 128. | Homo sapiens ATPase, Na+/K+ transporting, alpha 1 polypeptide (ATP1A1), |
| | transcript variant 1, mRNA. |
| 129. | Homo sapiens BAT2 domain containing 1 (BAT2D1), mRNA. |
| 130. | Homo sapiens small nuclear ribonucleoprotein polypeptide N (SNRPN), |
| | transcript variant 2, mRNA. |
| 131. | Homo sapiens zinc finger, matrin type 5 (ZMAT5), transcript variant 1, |
| | mRNA. |
| 132. | Homo sapiens SAPK substrate protein 1 (LOC51035), mRNA. |
| 133. | Homo sapiens vacuolar protein sorting 26 homolog B (S. pombe) (VPS26B), |
| | mRNA. |
| 134. | Homo sapiens catenin (cadherin-associated protein), beta 1, 88 kDa (CTNNB1), |
| | mRNA. |
| 135. | Homo sapiens DCN1, defective in cullin neddylation 1, domain containing 5 |
| | (S. cerevisiae) (DCUN105), mRNA. |
| 136. | Homo sapiens DEAH (Asp-Glu-Ala-His) box polypeptide 15 (DHX15), |
| | mRNA. |
| 137. | Homo sapiens hydroxysteroid (17-beta) dehydrogenase 4 (HSD17B4), mRNA. |
| 138. | Homo sapiens 6-phosphofructo-2-kinase/fructose-2,6-biphosphatase 3 |
| | (PFKFB3), mRNA. |
| 139. | Homo sapiens 5′-nucleotidase domain containing 2 (NT5DC2), mRNA. |
| 140. | Homo sapiens eukaryotic translation initiation factor 4 gamma, 2 (EIF4G2), |
| | transcript variant 1, mRNA. |
| 141. | Homo sapiens mitochondrial ribosomal protein S30 (MRPS30), nuclear gene |
| | encoding mitochondrial protein, mRNA. |
| 142. | Homo sapiens choline phosphotransferase 1 (CHPT1), mRNA. |
| 143. | Homo sapiens ATP synthase, H+ transporting, mitochondrial F0 complex, |
| | subunit G (ATP5L), nuclear gene encoding mitochondrial protein, mRNA. |
| 144. | Homo sapiens casein kinase 2, beta polypeptide (CSNK2B), mRNA. |
| 145. | Homo sapiens MARCKS-like 1 (MARCKSL1), mRNA. |
| 146. | Homo sapiens programmed cell death 2 (PDCD2), transcript variant 1, mRNA. |
| 147. | Homo sapiens mitochondrial ribosomal protein L12 (MRPL12), nuclear gene |
| | encoding mitochondrial protein, mRNA. |
| 148. | Homo sapiens Yip1 interacting factor homolog A (S. cerevisiae) (YIF1A), |
| | mRNA. |
| 149. | Homo sapiens CKLF-like MARVEL transmembrane domain containing 8 |
| | (CMTM8), mRNA. |
| 150. | Homo sapiens histocompatibility (minor) 13 (HM13), transcript variant 2, |
| | mRNA. |
| 151. | Homo sapiens protein phosphatase 1, regulatory (inhibitor) subunit 14B |
| | (PPP1R14B), mRNA. |
| 152. | Homo sapiens interferon, alpha-inducible protein 27 (IFI27), mRNA. |
| 153. | Homo sapiens quinolinate phosphoribosyltransferase (nicotinate-nucleotide |
| | pyrophosphorylase (carboxylating)) (QPRT), mRNA. |
| 154. | Homo sapiens signal transducer and activator of transcription 3 (acute-phase |
| | response factor) (STAT3), transcript variant 3, mRNA. |
| 155. | Homo sapiens deoxyuridine triphosphatase (DUT), nuclear gene encoding |
| | mitochondrial protein, transcript variant 1, mRNA. |
| 156. | Homo sapiens CTD (carboxy-terminal domain, RNA polymerase II, |
| | polypeptide A) small phosphatase-like (CTDSPL), transcript variant 2, mRNA. |
| 157. | Homo sapiens chromosome 14 open reading frame 147 (C14orf147), mRNA. |
| 158. | Homo sapiens chromosome 14 open reading frame 124 (C14orf124), mRNA. |
| 159. | Homo sapiens zinc finger, MYND-type containing 8 (ZMYND8), transcript |
| | variant 1, mRNA. |
| 160. | Homo sapiens yippee-like 5 (Drosophila) (YPEL5), mRNA. |
| 161. | Homo sapiens gap junction protein, gamma 1, 45 kDa (GJC1), transcript variant |
| | 1, mRNA. |
| 162. | Homo sapiens nicolin 1 (NICN1), mRNA. |
| 163. | Homo sapiens squalene epoxidase (SQLE), mRNA. |
| 164. | Homo sapiens chromosome 6 open reading frame 130 (C6orf130), mRNA. |
| 165. | Homo sapiens penta-EF-hand domain containing 1 (PEF1), mRNA. |
| 166. | Homo sapiens bolA homolog 2 (E. coli) (BOLA2), mRNA. |
| 167. | Homo sapiens phosphatidylinositol transfer protein, membrane-associated 1 |
| | (PITPNM1), mRNA. |
| 168. | Homo sapiens growth arrest-specific 6 (GAS6), mRNA. |
| 169. | Homo sapiens disabled homolog 2, mitogen-responsive phosphoprotein |
| | (Drosophila) (DAB2), mRNA. |
| 170. | Homo sapiens nuclear prelamin A recognition factor (NARF), transcript |
| | variant 3, mRNA. |
| 171. | Homo sapiens receptor accessory protein 5 (REEP5), mRNA. |
| 172. | Homo sapiens ADP-ribosylation factor GTPase activating protein 3 |
| | (ARFGAP3), mRNA. |
| 173. | Homo sapiens 5′-nucleotidase, cytosolic III (NT5C3), transcript variant 1, |
| | mRNA. |
| 174. | Homo sapiens polymerase I and transcript release factor (PTRF), mRNA. |
| 175. | Homo sapiens mediator complex subunit 20 (MED20), mRNA. |
| 176. | Homo sapiens chromosome 8 open reading frame 33 (Caort33), mRNA. |
| 177. | Homo sapiens protein kinase C and casein kinase substrate in neurons 2 |
| | (PACSIN2), mRNA. |
| 178. | Homo sapiens ribosomal protein S23 (RPS23), mRNA. |
| 179. | Homo sapiens signal-induced proliferation-associated gene 1 (SIPA1), |
| | transcript variant 2, mRNA. |
| 180. | Homo sapiens EH domain binding protein 1 (EHBP1), mRNA. |
| 181. | Homo sapiens tight junction associated protein 1 (peripheral) (TJAP1), mRNA. |
| 182. | PREDICTED: Homo sapiens similar to tropomyosin 3 isoform 2 |
| | (LOC644330), mRNA. |
| 183. | Homo sapiens Wolfram syndrome 1 (wolframin) (WFS1), mRNA. |
| 184. | Homo sapiens GIPC PDZ domain containing family, member 1 (GIPC1), |
| | transcript variant 3, mRNA. |
| 185. | Homo sapiens thioredoxin domain containing 5 (TXNDC5), transcript variant |
| | 2, mRNA. |
| 186. | Homo sapiens transforming growth factor, beta receptor II (70/80 kDa) |
| | (TGFBR2), transcript variant 1, mRNA. |
| 187. | Homo sapiens retinoic acid induced 14 (RAI14), mRNA. |
| 188. | Homo sapiens CD81 molecule (CD81), mRNA. |
| 189. | Homo sapiens cytidine monophosphate N-acetylneuraminic acid synthetase |
| | (CMAS), mRNA. |
| 190. | Homo sapiens ATG4 autophagy related 4 homolog B (S. cerevisiae) (ATG4B), |
| | transcript variant 2, mRNA. |
| 191. | Homo sapiens inhibitor of kappa light polypeptide gene enhancer in B-cells, |
| | kinase gamma (IKBKG), transcript variant 2, mRNA. |
| 192. | Homo sapiens podocalyxin-like (PODX L), transcript variant 1, mRNA. |
| 193. | Homo sapiens chromosome 14 open reading frame 173 (C14orf173), transcript |
| | variant 2, mRNA. |
| 194. | Homo sapiens transforming growth factor, beta-induced, 68 kDa (TGFBI), |
| | mRNA. |
| 195. | Homo sapiens cyclin D3 (CCND3), mRNA. |
| 196. | Homo sapiens basic helix-loop-helix domain containing, class B, 2 (BHLHB2), |
| | mRNA. |
| 197. | Homo sapiens NADH dehydrogenase (ubiquinone) 1 beta subcomplex, 3, |
| | 12 kDa (NDUFB3), mRNA. |
| 198. | Homo sapiens ferritin, heavy polypeptide-like 7 (FTHL7) on chromosome 13. |
| 199. | Homo sapiens translocator protein (18 kDa) (TSPO), transcript variant PBR, |
| | mRNA. |
| 200. | Homo sapiens discoidin, CUB and LCCL domain containing 2 (DCBLD2), |
| | mRNA. |
| 201. | Homo sapiens fer-1-like 3, myoferlin (C. elegans) (FER1L3), transcript variant |
| | 2, mRNA. |
| 202. | PREDICTED: Homo sapiens similar to Heterogeneous nuclear |
| | ribonucleoprotein A1 (Helix-destabilizing protein) (Single-strand RNA- |
| | binding protein) (hnRNP core protein A1) (HDP) (LOC648210), mRNA. |
| 203. | Homo sapiens chromosome 20 open reading frame 24 (C20orf24), transcript |
| | variant 2, mRNA. |
| 204. | Homo sapiens cathepsin B (CTSB), transcript variant 1, mRNA. |
| 205. | Homo sapiens ribosomal protein L36a-like (RPL36AL), mRNA. |
| 206. | Homo sapiens reticulon 4 (RTN4), transcript variant 3, mRNA. |
| 207. | Homo sapiens adrenomedullin (ADM), mRNA. |
| 208. | Homo sapiens hippocalcin-like 1 (HPCAL1), transcript variant 2, mRNA. |
| 209. | Homo sapiens interleukin 8 (IL8), mRNA. |
| 210. | Homo sapiens actin, beta (ACTB), mRNA. |

### Example 2

### Gene Array Validation with Antibody Microarray—Methods

Tumor cell implantation (Table 2). Internal carotid (IC) injection. Prior to internal carotid injection, nude mice were anesthetized by the intraperitoneal injection of Nembutal (0.5 mg/g body weight, Abbott Laboratories, North Chicago, Ill.). Cells were harvested by brief exposure to 0.02% EDTA-0.25% Trypsin and resuspended in Mg⁺⁺/Ca⁺⁺ free HBSS. Cells were injected into the brain of the mice through the internal carotid artery. In brief, the anesthetized mice were put in the supine position on a glass plate and placed under a dissecting microscope. The neck was prepared for surgery by swabbing with alcohol-iodine and the skin was cut by a midline incision. The trachea was exposed by blunt dissection, the muscles were separated to expose the common carotid artery, and then internal and external carotid arteries were identified. The artery was prepared for injection distal to the bifurcation of the internal and external carotid arteries. A ligature of 6-0 black silk was placed around the distal part of the common carotid artery, a second ligature was placed proximal to the injection site and the external carotid artery was ligated. Injection was made by a 30G needle and placed ligatures were tied. Performance status (movement and especially eating) was closely observed because increased intracranial pressures (IICP) in mice lead to poor ambulation and food/water intake, which is related to post-injection mortality from dehydration. Close surveillance was continued until food/water intake normalized.

Mammary fat pad (MFP) injection. Prior to mammary fat pad injection, nude mice were anesthetized by the intraperitoneal injection of Nembutal (0.5 mg/g body weight, Abbott Laboratories, North Chicago, Ill.). Cells were harvested from subconfluent cultures by a brief exposure to 0.25% trypsin and 0.02% EDTA. Trypsinization was stopped with medium containing 10% fetal bovine serum, and the cells were washed once in serum-free medium and resuspended in Mg⁺⁺/Ca⁺⁺ free HBSS. Tumor cells were suspended in 50 μL HBSS and implanted under the MFP of mice. Tumor sizes in this model can be assessed either by BLI or caliper measurements.

Intratibial (bone) injection. To produce bone tumors, cells were harvested from subconfluent cultures by a brief exposure to 0.25% trypsin and 0.02% EDTA. Trypsinization was stopped with medium containing 10% fetal bovine serum, and the cells were washed once in serum-free medium and resuspended in Mg⁺⁺/Ca⁺⁺ free HBSS. Cell viability was determined by trypan blue exclusion, and only single-cell suspensions of 95% viability were used to produce tumors in the tibia of mice. Nude mice were anesthetized with Nembutal (Abbott Laboratories, North Chicago, Ill.). A percutaneous intraosseal injection was made by drilling a 27-gauge needle into the tibia immediately proximal to the tuberositas tibia. After penetration of the cortical bone, the needle was inserted into the shaft of the tibia, and 20 μl of the cell suspension were deposited in the bone cortex. To prevent leakage of cells into the surrounding muscles, a cotton swab was held for 1 min over the site of injection. The animals tolerated the surgical procedure well, and no anesthesia-related deaths occurred.

Stereotactic (ST) injection. Prior to stereotactic cell injection, nude mice were anesthetized by the intraperitoneal injection of Nembutal (0.5 mg/g body weight, Abbot Laboratories, North Chicago, Ill.). Cells were harvested by brief exposure to 0.02% EDTA-0.25% Trypsin and re-suspended in Mg⁺⁺/Ca⁺⁺ free HBSS. Cells were stereotactically injected into the brain of the mice. In brief, the nude mouse was fixed in the stereotactic (x-yz) frame and a single incision was made from the anterior pole of the skull to the posterior ridge (7-8 mm). A hole was drilled over the target area and cells were injected using the following stereotactic coordinates, all relative to the bregma: 1.5 mm rostral, 1.5 mm anterior and 4 mm below the pial surface. An automated micro-pump (Stoelting Instruments, Wood Dale, Ill.) was used to dispense cells in 4 μL cell suspension over a 2 minute period. After injection, the hole was plugged with bone wax and skin was closed with skin staples. After injection, the mouse was placed on a heating pad until recovery from the anesthesia. Performance status (movement and, especially, eating) was closely observed because increased intracranial pressure (IICP) in mice leads to poor ambulation and food/water intake, which is related to post-injection mortality from dehydration. Close surveillance was continued until food/water intake normalized.

To produce GBM tumors mice were injected stereotactically with 100,000 viable LN 229-Luc (Human Glioblastoma) cells. Tumor establishment and growth was monitored by IVIS imaging.

TABLE 2
Tumor cell implantation.

| Cell line | Primary tumor | Brain Metastasis |
|---|---|---|
| MDA231-Luc | MFP | IC |
| | 100,000 cells | 5,000 cells |
| PC14-Luc | | IC |
| | | 5,000 cells |
| LN229-Luc | Brain (ST) | — |
| | 100,000 cells | |

IVIS imaging. Mice were imaged every week after implantation of the tumors by IVIS100.

Serum preparation from mice. Tumor harboring mice were maintained under general (inhalant) anesthesia. A 30G needle was inserted into the left ventricle of the heart by entering the left second intercostal space and proceeding anteromedially through the left margin of the sternum and then up to 300 μl of blood was withdrawn from the heart. The needle was quickly removed from heart and then from the syringe, and blood was emptied into a plain 1.5 mL microcentrifuge tube. After collection of the blood, the blood was allowed to clot by leaving it undisturbed at room temperature for 1 hour. The clot was spun-down by centrifuging at 1,000-2,000×g for 10 minutes in a refrigerated centrifuge. After centrifugation, the supernatant was transferred to new tubes, and stored at −20° C. or lower.

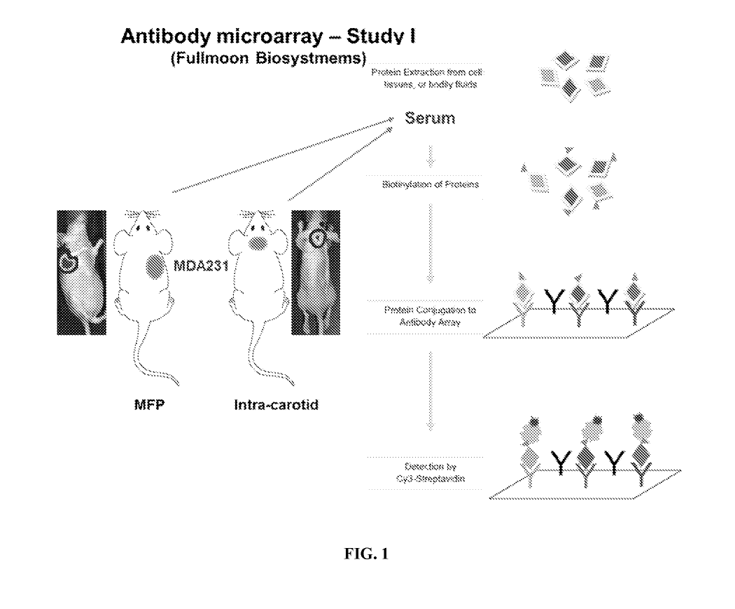

Antibody microarray. Protein expression levels were examined using the Full Moon BioSystems Explorer Antibody Microarray (Cat. no. ASB600) and Antibody Array Assay Kit (Cat. No. KAS20), according to the manufacturer's specifications (Full Moon BioSystems, Inc.). An example illustration of the procedure is shown in FIG. 1.

A. Protein Labeling—Biotinylation of Protein Samples
1. Biotin Preparation
   a. Briefly centrifuge Biotin Reagent before use.
   b. Add 100 μl of DMF (N,N-Dimethylformamide) to 1 mg of Biotin Reagent to give a concentration of 10 μg/μl. Label this solution as Biotin/DMF.
2. Labeling
   a. Aliquot 3-4 μl of serum.
   b. Add Labeling Buffer to the protein sample to bring the volume to 75 μl.
   c. Add 3 μl of the Biotin/DMF solution to the protein sample. Incubate the mixture at room temperature for 1-2 hours with mixing.
   d. Add 35 μl of Stop Reagent. Incubate for 30 minutes at room temperature with mixing.
   e. Proceed immediately to the next step, or store the sample at −80° C. for future use.

B. Blocking
1. Add 30 ml of Blocking Solution in a 100×15 mm Petri dish.
2. Submerge one slide in the Blocking Solution. The side with a barcode label must face up.
3. Incubate on an orbital shaker rotating at 55 rpm for 30-45 minutes at room temperature.
4. Rinse the slide extensively with Milli-Q grade water.

C. Coupling
1. In a tube, add 6 ml of Coupling Solution.
2. Add one tube of biotin labeled proteins (80-150 OD or 40-100 μg). Vortex briefly to mix. Label it as “Protein Coupling Mix.”
3. Place the slide in Well 1 (or any clean well) of the Coupling Chamber.
4. Slowly pour all 6 ml of Protein Coupling Mix over the slide.
5. Incubate on an orbital shaker rotating at 35 rpm for 1-2 hours at room temperature.
6. Transfer the slide to a 100×15 mm Petri dish containing 30 ml of 1× Wash Solution.
7. Incubate on an orbital shaker rotating at 55 rpm for 10 minutes at room temperature. Discard the wash solution. Repeat the wash step twice.
8. Rinse the slide extensively with Milli-Q grade water.
9. Shake off excessive water on the slide surface and proceed to the next step immediately.

D. Detection
1. Add 60 μl of Cy3-Streptavidin (0.5 mg/ml) to 60 ml of Detection Buffer.
2. Pour 30 ml of Cy3-Streptavidn Solution into a 100×15 mm Petri dish.
3. Submerge the slide in the Cy3-Streptavidin solution. Incubate on an orbital shaker rotating at 55 rpm for 20 minutes at room temperature in the dark or covered with aluminum foil.
4. Transfer the slide to a new 100×15 mm Petri dish containing 30 ml of 1× Wash Solution.
5. Incubate on an orbital shaker set at 55 rpm for 10 minutes at room temperature. Discard the wash solution. Repeat the wash step twice.
6. Rinse the slide extensively with Milli-Q grade water.
7. Hold the slide with your fingers, shake off excess water from the slide.
8. Dry the slide with compressed nitrogen (or air) or by centrifugation.
9. The slide is now ready for scanning.

### Example 3

### Gene Array Validation with Antibody Microarray—Results

Study 1. Tumor cells (MDA-231 breast cancer cells) were implanted into mice by either mammary fat pad injection or internal carotid injection. Mice were imaged (FIG. 1) and serum was collected for Explorer antibody microarray analysis (Table 3). Most of the proteins in the gene array list were up-regulated in brain metastasis.

TABLE 3
Explorer Array analysis.

| | | * Sign fold changes |
|---|---|---|
| symbol | Explorer Array | MDA - MFP vs. IC |
| GRIA2 | (GluR2/3) | 0.20 |
| C12orf48 | (MCH) | 0.04/−0.33 |
| GRM3 | (GluR2/3) | 0.20 |
| CRYAB | (Crystallin, AB) | 0.04 |
| CELSR3 | (cadherin) | −0.02 |
| SREBF1 | O | 0.22 |
| LOC100008589 | (CDC14A phosphatase) | −0.04 |
| CTNNB1 | (beta-catenin) | 0.04 |
| STAT3 | O | 0.01 |
| CD81 | O | 0.07 |
| FTHL7 | (ferritin) | −0.06 |
| IL8 | O | 0.14 |
| ACTB | O | 0.16 |

* Proteins marked with italicized numbers (fold changes) are detectable in serum.

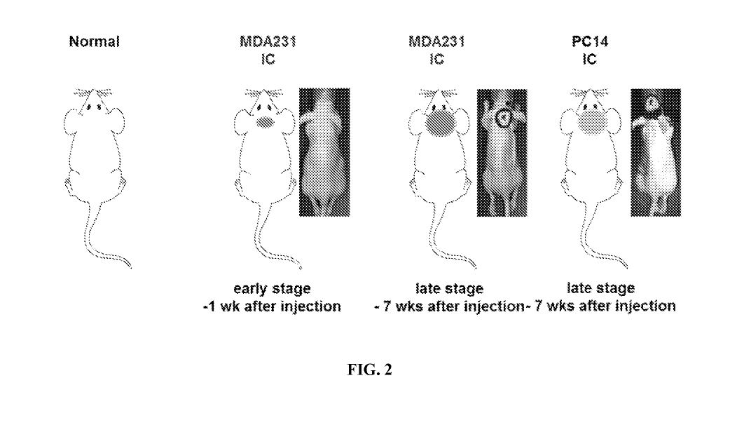

Study 2. Tumor cells (MDA-231 breast cancer cells or PC14 lung adenocarcinoma cells) were implanted into mice by internal carotid injection. Mice were imaged (FIG. 2) and serum was collected either 1 week or 7 weeks after injection. Serum was analyzed using the Explorer antibody microarray (Table 4).

TABLE 4
Normal/MDA231 - IC early, late. Normal/PC14- IC early, late.

| | | Normal vs. | Normals vs. | Normal vs. | MDA-IC |
|---|---|---|---|---|---|
| | | MBA-early | MDA-late | PC14-late | early vs. |
| SYMBOL | Explorer Array | brain | brain | brain | late brain |
| GRIA2 | (GluR2/3) | −0.18 | −0.20 | 0.02 | −0.03 |
| C12orf48 | (MCH) | −0.20 | −0.16 | −0.02 | 0.05 |
| | | 0.09 | −0.08 | −0.46 | −0.16 |
| GRM3 | (GluR2/3) | −0.18 | −0.20 | 0.02 | −0.03 |
| CRYAB | (Crystallin, AB) | 0.57 | 0.49 | 0.35 | −0.05 |
| CELSR3 | (cadherin-pan) | −0.31 | −0.42 | −0.21 | −0.16 |
| | (cadherin-E) | −0.31 | −0.37 | −0.28 | −0.09 |
| | (cadherin-P) | 0.40 | 0.39 | 0.46 | −0.01 |
| SREBF1 | O | 0.02 | 0.08 | 0.08 | 0.07 |
| LOC100008589 | (CDC14A | −0.13 | 0.28 | 0.07 | 0.48 |
| | phosphatase) | | | | |
| CTNNB1 | (beta-catenin) | −0.55 | −0.57 | −0.36 | −0.03 |
| STAT3 | O | −0.24 | −0.34 | −0.35 | −0.14 |
| CD81 | O | 0.02 | 0.33 | 0.11 | 0.31 |
| FTHL7 | (ferritin) | 0.26 | 1.19 | 0.15 | 0.75 |
| IL8 | O | 0.14 | 0.04 | −0.29 | −0.08 |
| ACTB | O | 0.07 | 0.44 | 0.38 | 0.35 |

Proteins marked with italicized numbers (fold changes) are detectable in serum.

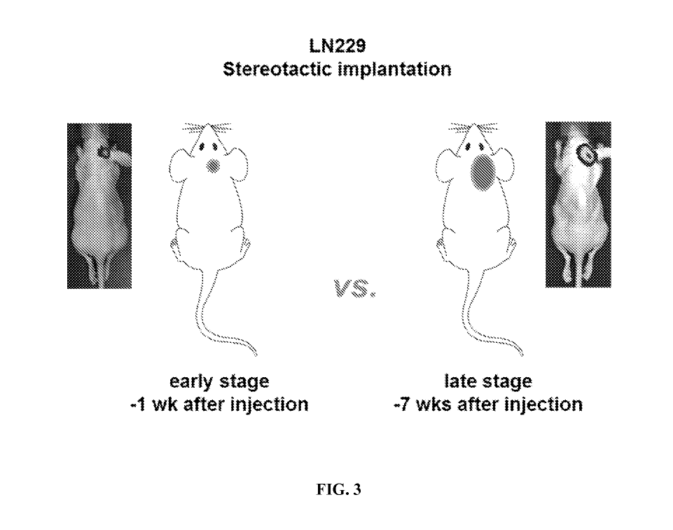

Study 3. Tumor cells (LN229 glioblastoma cells) were implanted into mice by stereotactic injection. Mice were imaged (FIG. 3) and serum was collected either 1 week or 7 weeks after injection. Serum was analyzed using the Explorer antibody microarray (Table 5).

TABLE 5
Analysis: LN229-LUC glioblastoma early vs. late.

| SYMBOL | Explorer Array | LN229-ST early vs. late |
|---|---|---|
| GRIA2 | (GluR2/3) | 0.39 |
| C12orf48 | (MCH2) | 0.19 |
| | (MCH3) | −0.32 |
| GRM3 | (GluR2/3) | 0.39 |
| CRYAB | (Crystallin, AB) | 0.12 |
| CELSR3 | (cadherin-pan) | 0.26 |
| | (cadherin-E) | 0.08 |
| | (cadherin-P) | 0.21 |
| SREBF1 | O | 0.23 |
| LOC100008589 | (CDC14A phosphatase) | 0.40 |
| CTNNB1 | (beta-catenin) | 0.16 |
| STAT3 | O | −0.07 |
| CD81 | O | −0.31 |
| FTHL7 | (ferritin) | 0.13 |
| IL8 | O | −0.24 |
| ACTB | O | −0.18 |

Proteins marked with italicized numbers (fold changes) are detectable in serum.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An in vitro method of detecting if a subject is at risk for a brain cancer or cancer brain metastasis, comprising:
(a) obtaining a biological sample from the subject;
(b) measuring the expression level of one or more genes in the sample selected from the group consisting of GRIA2, C12orf48, GRM3, CRYAB, CELSR3 (cadherin-P), SREBF1, LOC100008589, CTNNB1, STAT3, CD81, FTHL7, IL8, and ACTB; and
(c) identifying the subject as at risk or not at risk for a brain cancer or a cancer brain metastasis based on the expression level of said genes.

2. The method of claim 1, wherein the subject has or is diagnosed with a cancer.

3. The method of claim 2, wherein the cancer is a breast cancer, lung cancer, head & neck cancer, prostate cancer, esophageal cancer, tracheal cancer, brain cancer, liver cancer, bladder cancer, stomach cancer, pancreatic cancer, ovarian cancer, uterine cancer, cervical cancer, testicular cancer, colon cancer, rectal cancer or skin cancer.

4. The method of claim 1, wherein the subject is identified as at risk for a brain cancer or cancer brain metastasis if the expression of one or more of the genes is elevated in the sample as compared to a reference.

5. The method of claim 1, further comprising measuring the expression level of at least 2 of said genes, wherein elevated expression of the genes compared to a reference indicates that the subject as at risk for a cancer brain metastasis.

6. The method of claim 1, further comprising measuring the expression level of at least 3, 4, 5, 6, 7, 8, 9, 10, 11 or 12 of said genes.

7. The method of claim 1, wherein the sample is a blood sample.

8. The method of claim 1, further comprising measuring the expression level of two, three or more of the CRYAB, CELSR3, SREBF1, CD81, FTHL7, ACTB, C12orf48, LOC100008589, or IL8 genes.

9. The method of claim 1, further comprising measuring the expression level of two, three or more of the CRYAB, CELSR3, SREBF1, CD81, FTHL7, or ACTB genes.

10. The method of claim 1, wherein the subject has previously been treated for a cancer.

11. The method of claim 10, wherein the subject has previously had a tumor surgically removed.

12. The method of claim 1, wherein identifying the subject as at risk or not at risk for a brain cancer or cancer brain metastasis further comprises correlating the measured expression level(s) with a risk for cancer brain metastasis.

13. The method of claim 1, wherein identifying the subject as at risk or not at risk for a cancer brain metastasis further comprises analysis of the measured expression level(s) using an algorithm.

14. The method of claim 13, wherein said analysis is performed by a computer.

15. The method of claim 1, further comprising:
(b) measuring the expression level of the gene(s) in the sample and measuring the expression level of the genes in a reference sample; and
(c) identifying the subject as at risk or not at risk for a brain cancer or cancer brain metastasis by comparing the expression level of the gene(s) in the sample from the subject to the expression level of the genes in the reference sample.

16. The method of claim 1, wherein measuring expression of said gene(s) comprises measuring protein expression levels.

17. The method of claim 16, wherein measuring protein expression levels comprises performing an ELISA or binding to an antibody array.

18. The method of claim 1, wherein the measuring expression of said genes comprises measuring RNA expression levels.

19. The method of claim 18, wherein measuring RNA expression levels comprises performing RT-PCR, Northern blot or an array hybridization.

20. The method of claim 1, further comprising reporting whether the subject is at risk or not at risk for a brain cancer or cancer brain metastasis.

21. The method of claim 20, wherein reporting comprises preparing a written or electronic report.

22. The method of claim 20, further comprising providing the report to the patient, a doctor, a hospital or an insurance company.

23. A method of treating a subject comprising:
selecting a subject identified as at risk for a brain cancer or cancer brain metastasis in accordance with claim 1; and
administering an anti-cancer therapy the subject.

24. The method of claim 23, wherein the anti-cancer therapy is a chemotherapy, a radiation therapy, a hormonal therapy, a targeted therapy, an immunotherapy or a surgical therapy.

25. The method of claim 23, wherein the anti-cancer therapy is targeted to the brain.

26. A method of treating a subject comprising:
(a) obtaining the expression level of one or more genes selected from the group consisting of GRIA2, C12orf48, GRM3, CRYAB, CELSR3 (cadherin-P), SREBF1, LOC100008589, CTNNB1, STAT3, CD81, FTHL7, IL8, and ACTB in a sample from the subject;
(b) selecting a subject having a risk for a brain cancer or cancer brain metastasis based on the expression level of said gene(s); and
(c) treating the selected subject with an anti-cancer therapy.

27. The method of claim 26, wherein the anti-cancer therapy is a chemotherapy, a radiation therapy, a hormonal therapy, a targeted therapy, an immunotherapy or a surgical therapy.

28. The method of claim 26, wherein the anti-cancer therapy is targeted to the brain.

29. A method of selecting a subject for a diagnostic procedure comprising:
(a) obtaining the expression level of one or more genes selected from the group consisting of GRIA2, C12orf48, GRM3, CRYAB, CELSR3 (cadherin-P), SREBF1, LOC100008589, CTNNB1, STAT3, CD81, FTHL7, IL8, and ACTB in a sample from the subject;
(b) selecting a subject having a risk for a brain cancer or cancer brain metastasis based on the expression level of said gene(s); and
(c) performing a diagnostic procedure on the subject on the subject.

30. The method of claim 29, wherein the diagnostic procedure comprises imaging of the head.

31. The method of claim 30, wherein the imaging is a X-ray, CT, MRI or PET imaging.

32. A tangible computer-readable medium comprising computer-readable code that, when executed by a computer, causes the computer to perform operations comprising:
a) receiving information corresponding to a level of expression of GRIA2, C12orf48, GRM3, CRYAB, CELSR3 (cadherin-P), SREBF1, LOC100008589, CTNNB1, STAT3, CD81, FTHL7, IL8, or ACTB gene in a sample from a subject; and
b) determining a relative level of expression of one ore more of said genes compared to a reference level, wherein altered expression of one ore more of said genes compared to a reference level indicates that the subject is at risk of having a brain cancer or cancer brain metastasis.

33. The tangible computer-readable medium of claim 32, further comprising receiving information corresponding to a reference level of expression of GRIA2, C12orf48, GRM3, CRYAB, CELSR3 (cadherin-P), SREBF1, LOC100008589, CTNNB1, STAT3, CD81, FTHL7, IL8, or ACTB in a sample from a subject without brain metastasis.

34. The tangible computer-readable medium of claim 32, wherein the reference level is stored in said tangible computer-readable medium.

35. The tangible computer-readable medium of claim 32, wherein the receiving information comprises receiving from a tangible data storage device information corresponding to a level of expression of one or more of said gene in a sample from a subject.

36. The tangible computer-readable medium of claim 32, further comprising computer-readable code that, when executed by a computer, causes the computer to perform one or more additional operations comprising: sending information corresponding to the relative level of expression of one or more of said genes to a tangible data storage device.

37. The tangible computer-readable medium of claim 32, wherein the receiving information further comprises receiving information corresponding to a level of expression of at least 2, 3, 4, 5, 6, 7, 8, 9, 10, 11 or 12 of said genes in a sample from a subject.

38. The tangible computer-readable medium of claim 32, wherein the computer-readable code, when executed by a computer, causes the computer to perform operations further comprising: c) calculating a diagnostic score for the sample, wherein the diagnostic score is indicative of the probability that the sample is from a subject having a brain cancer or cancer brain metastasis.
